(19) United States
(12) Patent Application Publication
(10) Pub. No.: US 2014/0171963 A1
(43) Pub. Date: Jun. 19, 2014

(54) METHOD AND DEVICE FOR SELECTIVE APPLICATION OF A LIQUID JET

(71) Applicant: Deutsches Zentrum fur Luft-und Raumfahrt E.V., Cologne (DE)

(72) Inventors: Thomas Bahls, Weil (DE); Florian Alexander Froehlich, Germering (DE)

(73) Assignee: Deutsches Zentrum fur Luft-und Raumfahrt E.V., Cologne (DE)

(21) Appl. No.: 13/961,036

(22) Filed: Aug. 7, 2013

(30) Foreign Application Priority Data
Aug. 9, 2012 (DE) 102012214152.9

(51) Int. Cl. A61B 19/00; A61B 17/3203

(52) U.S. Cl. CPC A61B 19/2203; A61B 17/3203
USPC 606/130; 606/186

(57) ABSTRACT

A method for selectively applying a liquid jet onto and/or into human or animal skin is provided. The method includes the steps of detecting and storing a position, orientation and shape of a skin patch to be processed and moving a liquid nozzle over the skin patch. The liquid nozzle is guided by a robot arm within boundaries of the skin patch and the liquid jet is applied in a pulsed manner into and/or onto the skin. At each spot of the skin patch to be processed at each point of time in processing only one liquid jet pulse each is applied and the next pulse is applied to another spot of the skin patch to be processed to prevent accumulation of liquid in the skin to thus attain penetration of the liquid jet.

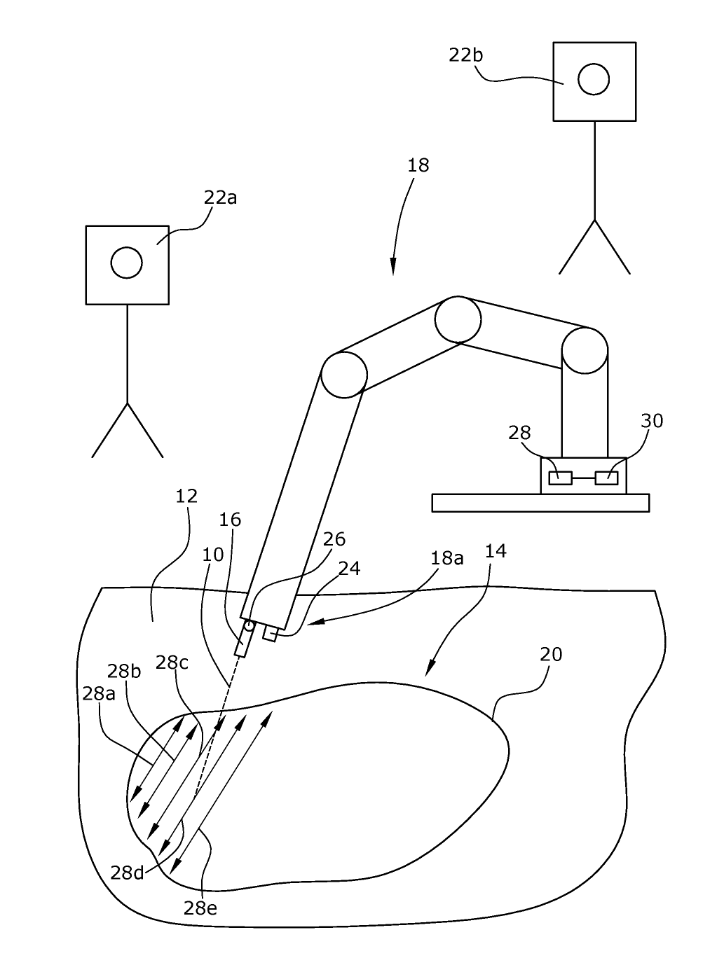

### BACKGROUND

1. Field of the Disclosure

The present disclosure relates to a method and a device for selective application of a liquid jet onto and/or into human or animal skin.

2. Discussion of the Background Art

The application and removal of tattoos to and/or from the skin is a difficult task which can be carried out only by well trained persons. So far, the aforementioned operations have been carried out manually such that the accuracy in preparation of a tattoo or removal of a tattoo has been depending on the skills of the person carrying out said operations.

### SUMMARY

It is an object of the present disclosure to provide a method with the aid of which a liquid jet, in particular tattooing ink or water for removing a tattoo, can be selectively applied onto and/or into the skin.

In the method according to the disclosure, a liquid jet is selectively applied onto and/or into the human or animal skin. The method comprises the following steps:

a) First, the position, orientation and shape of the skin patch to be processed are detected and stored. This is necessary to obtain detailed knowledge of the geometrical properties of the skin patch to be processed such that the latter can subsequently be precisely worked on. For this purpose it is possible to first detect the geometrical properties of the body part to be processed. To this end it is e. g. possible to scan the outer contour of the body part. If the tattoo is to be removed, it is further possible, in a subsequent process step, to detect and determine the skin patch to be processed using e. g. an image processing software. For this purpose, the texture of the tattoo which differs from the texture of the remaining skin can be automatically recognized.

b) In particular, a liquid nozzle guided by a robot arm is repetitively moved over the skin patch to be processed. Within the boundaries of the skin patch to be processed, whose position, orientation and shape are known, the liquid jet is preferably applied to the skin patch in an oscillating movement. Oscillating in this connection means that the liquid jet reciprocates at a constant or varying velocity within the boundaries of the skin patch to be processed, and thus the overall surface of the skin patch to be processed is gradually worked on without the liquid jet dwelling in one position for an extended period of time. Repetitive means that each spot of the skin patch to be processed is not processed only once but several times by the liquid jet. Instead of an oscillating movement the liquid jet can be moved over the skin patch to be processed in any other movement pattern. It is merely of importance that each spot of the skin patch to be processed is worked on to a sufficient extent. The liquid jet is introduced into a specific spot of the skin merely for a predetermined maximum time such that an injury of the skin can be prevented. Further, an accumulation of liquid in the skin can be prevented.

According to the disclosure, the liquid is applied in a pulsed manner into and/or onto the skin. Further, according to the disclosure, only one liquid jet pulse each is applied to each spot of the skin patch to be processed at each point of time in processing, and the next pulse is applied to another spot of the skin patch to be processed to prevent liquid from accumulating in the skin. Said accumulation would occur if the liquid jet pulse is too long and thus the liquid is introduced into a spot of the skin for an extended period of time. Then an accumulation or reflow of liquid occurs which produces a counter pressure such that the liquid jet cannot penetrate deeply enough into the skin. The aforementioned process steps allow the described liquid accumulation to be prevented and thus a deeper penetration of the liquid jet into the skin to be achieved. Repeated processing of the same spot is carried out only during a repetitive procedure if the first application of the liquid has not produced the desired result.

The liquid to be applied onto and/or into the skin may be tattooing ink such that the process is a method for a robot-assisted application of a tattoo onto the skin. In this case, the tattoo to be applied must be submitted beforehand in digital form and made available to the control unit of the robot such that the robot arm is driven in such a manner that the tattoo is introduced into the skin in accordance with the submitted design. Corresponding control of the pressure of the liquid jet allows the tattooing ink to be introduced into the skin, which is also the case when tattooing with the aid of needles. In all embodiments of the disclosure it is essential that the liquid itself is pressurized before being introduced into the skin. An advantage of the method according to the disclosure is the high precision which can be attained by the robot-assisted guiding of the liquid nozzle. It is thus possible to produce complex tattoos without a particularly skilled tattooist being required for this purpose.

Alternatively, the liquid to be applied onto and/or into the skin may be water or any other liquid which is introduced into the skin in a robot-assisted manner to remove a tattoo. In this embodiment, too, high precision can be attained by the robot-assisted introduction of the liquid.

In this embodiment it is preferred that the geometric configuration of the tattoo to be removed is automatically optically detected on the skin and stored, and the water jet for removing the tattoo is exclusively applied to skin patches containing tattooing ink. An injury of the other skin patches which need not be processed can thus be prevented. In prior art methods for removing tattoos such selective processing of dermal tissue is not possible.

Preferably, the position, orientation and shape of the skin patch to be processed is dynamically detected, i. e. also during application of the liquid into and/or onto the skin, such that a change in the orientation of the skin can be detected. Driving the robot arm is then correspondingly adapted to the changed position, orientation and/or shape of the skin patch to be processed.

The detection of the position, orientation and shape of the skin patch to be processed in accordance with process step a) can be carried out by preparing and storing a digital model of the skin patch to be processed.

The aforementioned information can be detected by scanning the skin patch to be processed with the aid of the distal end of the robot arm, wherein the respective spatial position of this spot is stored when the skin patch is touched by the distal end of the robot arm. This information can be used for the aforementioned modeling.

Alternatively, a model of the skin patch to be processed can be prepared in any other manner.

The aforementioned information on the skin patch to be processed can be used for placing the liquid nozzle exactly in the normal direction, i. e. locally perpendicularly to the skin surface. Thus an exactly defined liquid pressure can be constantly applied into the skin.

Preferably, the position of the liquid nozzle is detected in particular by an optical tracking system. This is realized during operation such that it is known at any time at which position exactly the liquid nozzle is located. On the basis of this information a position control of the liquid nozzle can be carried out.

Preferably, the contact pressure at which the liquid nozzle is pressed onto the skin is measured and controlled by a sensor. It is thus possible to constantly press the liquid nozzle to the skin at a predetermined maximum force. The maximum force can e. g. be predetermined according to implementation or adjusted by the person carrying out the treatment. Further, it is thus possible to compensate for inaccuracies in the surface model of the skin patch to be treated, which would lead to excessively firm placing of the nozzle. Hereby injuries of the tissue can be prevented. Further, an excessively weak contact or no contact at all with the surface can be prevented. In this case the method according to the disclosure would have a poor effect or no effect at all.

In a preferred embodiment the liquid is applied in a pulsed manner into and/or onto the skin, wherein the pulse duration ranges between 10 and 200 msec. The pressure applied lies in a range of 20 to 200 bar, preferably 30 to 100 bar, and particularly preferably 50 to 80 bar.

The method according to the disclosure is preferably suitable for other cosmetic applications where it is of importance to apply a liquid jet with high accuracy and at a defined pressure into and/or onto the skin. Medical applications, in particular surgical or therapeutic methods used on human and/or animal bodies, do not fall under the method according to the disclosure.

Preferably, following a first, in particular repetitive movement of the liquid nozzle over the skin patch to be processed in accordance with the above method step b), the geometrical configuration of the tattoo to be removed is again automatically optically detected and stored. Subsequently, the robot arm is again driven on the basis of this data such that the water jet for removing the tattoo is exclusively applied to skin patches still containing tattooing ink.

Further, the disclosure relates to a robot for selectively applying a liquid jet onto and/or into human or animal skin. The robot comprises a robot arm at whose distal end a liquid nozzle is attached. Further, a storage device is provided which is configured for storing the position, orientation and shape of the skin patch to be processed. The robot further comprises a control device for controlling the movement of the robot arm, said control device being configured to output control commands in particular for repetitively moving the robot arm over the skin patch to be processed and for simultaneously controlling the liquid nozzle such that within the boundaries of the skin patch to be processed, whose position, orientation and shape are known, the liquid jet is applied to the skin patch.

### BRIEF DESCRIPTION OF THE DRAWINGS

The sole FIGURE is a schematic representation of a robot according to the present disclosure.

### DETAILED DESCRIPTION OF THE PREFERRED EMBODIMENT

Hereunder an embodiment of the disclosure is described in detail with reference to the drawing.

The FIGURE shows the robot 18 according to the disclosure, at whose distal end 18a a liquid nozzle 16 is attached. Further, the distal end 18a may have attached thereto a measuring device 24 with the aid of which the skin patch 14 to be processed can be detected in a three-dimensional manner. Said detection can be carried out for modeling and planning purposes prior to application of the liquid and subsequently during operation by continuously detecting the position, orientation and shape of the skin patch 14 to be processed at a sufficiently high clock rate. In this connection it is sufficient to detect the aforementioned information with respect to the distal end 18a of the robot arm 18. The measuring device 24 may be e. g. a stereo camera, a PMD-sensor and a camera, a light section sensor and a camera etc.

The exact operational area of the robot arm 18 on the skin 12, i. e. the skin patch 14 to be processed, can be defined by the person carrying out the treatment in a screen display. Alternatively it is possible to define the boundaries 20 of the skin patch 14 to be processed by an algorithm which extracts this information from the image data detected by the measuring device 24. This can e. g. be realized within the framework of image data processing for detecting the margins of a tattoo to be removed. The automatically defined working area can then be adapted and approved by the person carrying out the treatment.

Subsequently, the robot arm 18 is controlled by the control unit 30, which accesses the storage 28, in such a manner that a complete and precise application of liquid 10 into and/or onto the skin patch 14 to be processed is performed. Here, the water jet 10 always remains within the boundary 20.

Preferably, the liquid nozzle 16 is connected to the distal end 18a of the robot arm 18 via a hinge 26. Thus the nozzle 16 can be pivoted independently of the last element of the robot arm 18.

Hereby a complete coverage of the skin patch 14 to be processed can be achieved without a large traveling distance of the robot arm 18 being necessary. For example, the nozzle 16 can be sinusoidally pivoted at a high velocity about the hinge 16 such that at each robot position the liquid jet 10 sweeps over a complete line section 28 of the skin patch 14 to be processed. For example, processing of the skin patch 14 can start at line section 28a, then the robot arm 18 is pivoted a short distance such that the liquid jet processes line section 28b etc. Thus it is possible to process the overall surface of the target area within a shorter time.

Preferably, for removing a tattoo the liquid nozzle is placed immediately onto the skin patch 14 to be processed. Subsequently, the water jet 10 is activated at a high pressure for a short period of time. Then the nozzle is lifted and moved to the next contact site on the skin 12. This process is repeated until the overall target area 14 has been processed.

Although the disclosure has been described and illustrated with reference to specific illustrative embodiments thereof, it is not intended that the disclosure be limited to those illustrative embodiments. Those skilled in the art will recognize that variations and modifications can be made without departing from the true scope of the disclosure as defined by the claims that follow. It is therefore intended to include within the disclosure all such variations and modifications as fall within the scope of the appended claims and equivalents thereof.

1. A method for selectively applying a liquid jet onto and/or into human or animal skin, comprising the following steps:
a) detecting a position, orientation and shape of a skin patch to be processed, wherein this information is stored,
b) moving a liquid nozzle over said skin patch to be processed, said liquid nozzle being guided by a robot arm, wherein within boundaries of said skin patch to be processed, whose position, orientation and shape are known, said liquid jet is applied to said skin patch,
wherein said liquid is applied in a pulsed manner into and/or onto said skin, and
at each spot of said skin patch to be processed at each point of time in processing only one liquid jet pulse each is applied and the next pulse is applied to another spot of said skin patch to be processed to prevent accumulation of liquid in said skin to thus attain a deeper penetration of said liquid jet.

2. The method according to claim 1, wherein detection of the position, orientation and shape of the skin patch to be processed in accordance with method step a) is performed by preparing and storing a digital model of said skin patch to be processed.

3. The method according to claim 1, wherein detection of the position, orientation and shape of the skin patch to be processed in accordance with method step a) is performed by scanning said skin patch to be processed with the aid of the distal end of the robot arm, wherein upon contact of said skin patch with said distal end of said robot arm the respective spatial position of this spot is stored.

4. The method according to claim 1, wherein the contact force at which the liquid nozzle is pressed onto the skin is measured and controlled by a sensor.

5. The method according to claim 1, wherein the liquid to be applied onto and/or into the skin is tattooing ink such that the method is a method for a robot-assisted application of a tattoo to said skin.

6. The method according to claim 1, wherein the liquid to be applied onto and/or into the skin is water or any other liquid introduced into said skin in a robot-assisted manner for removing a tattoo.

7. The method according to claim 6, wherein the geometric configuration of the tattoo to be removed is automatically optically detected on the skin and stored, and the robot arm is controlled on the basis of this data such that the water jet for removing the tattoo is applied exclusively to skin patches containing tattooing ink.

8. The method according to claim 1, wherein the liquid is applied in a pulsed manner into and/or onto the skin with the pulse duration of the liquid jet ranging between 10 and 200 msec.

9. The method according to claim 7, wherein after a first, in particular repetitive moving of the liquid nozzle over the skin patch to be treated in accordance with method step b) the geometrical configuration of the tattoo to be removed is detected again on the skin and the water jet for removing the remaining tattoo is again applied exclusively to said skin patches still containing tattooing ink.

10. A robot for selectively applying a liquid jet onto and/or into human or animal skin, comprising
a robot arm at whose distal end a liquid nozzle is attached;
a storage device configured for storing a position, orientation and shape of a skin patch to be processed;
a control device for controlling movements of said robot arm, said control device being configured to issue control commands for moving said robot arm over said skin patch to be processed and for simultaneously controlling said liquid nozzle such that within boundaries of said skin patch to be processed, whose position, orientation and shape are known, said liquid jet is applied to said skin patch.
